(19) United States
(12) Patent Application Publication
(10) Pub. No.: US 2010/0330171 A1
(43) Pub. Date: Dec. 30, 2010

(54) Dietary Supplement for Eye Health

(76) Inventors: Jeffrey P. Gilbard, Weston, MA (US); Johanna M. Seddon, Boston, MA (US)

Correspondence Address: FISH & RICHARDSON P.C. (BO), P.O. BOX 1022, MINNEAPOLIS, MN, 55440-1022, US

(21) Appl. No.: 12/691,034

(22) Filed: Jan. 21, 2010

Related U.S. Application Data
(63) Continuation of application No. 12/042,854, filed on Mar. 5, 2008, now abandoned.

(51) Int. Cl. A61K 9/64; A61K 36/82; A61K 31/59; A61K 31/355; A61K 31/34; A61K 33/30; A61K 33/34; A61P 27/02

(52) U.S. Cl. 424/456; 424/729; 514/167; 514/458; 514/474; 424/641; 424/638

(57) ABSTRACT

Nutritional supplements for eye health comprising: an anti-oxidant component; an anti-inflammatory component; and an anti-angiogenic component. The supplements inhibit development and progression of a range of retinal diseases and degenerations, particularly AMD and diabetic eye disease, diabetic retinopathy and diabetic macular edema. The supplements include tocotrienol and green tea extract.

### CROSS-REFERENCE TO RELATED APPLICATION

This application is a continuation application of and claims priority to U.S. application Ser. No. 12/042,854, filed on Mar. 5, 2008. This application is hereby incorporated by reference.

### TECHNICAL FIELD

This invention is in the general field of dietary supplements to improve eye health, particularly to prevent, reduce, delay or reverse macular degeneration (including age-related macular degeneration (AMD)) and diabetic eye diseases.

### BACKGROUND

Macular degeneration, including age-related macular degeneration (AMD) and diabetic eye diseases, are the most common causes of irreversible blindness in older people in many countries around the world.

Some supplements for AMD have focused on the role of free radical formation and oxidative processes and on the anti-oxidant properties of various nutrients. See, for example, Seddon J, Chen C., “Epidemiology of age-related macular degeneration”, In: Ryan S J, (ed). Retina, 4th edition. Volume Two. Medical Retina. St. Louis: C. V. Mosby; 2006, chapter 58, pages 1017-1027.

One supplement in particular, termed AREDS (Age Related Eye Disease Supplement), contains vitamin C—500 milligrams; vitamin E 400 IU; beta-carotene 15 milligrams; zinc (as zinc oxide) 80 milligrams; and copper (as cupric oxide) 2 milligrams. Results of a scientific trial using that supplement are published in Arch Ophthalmol. 119:1439-1452 (2001). A follow-on study (AREDS 2) is designed to assess the effects on the progression to advanced AMD of oral supplementation of high doses of two macular xanthophylls (lutein and zeaxanthin) and omega-3 long-chain PUFAs (DHA and EPA). Participants will be offered additional treatment with the original AREDS formulation and three variations of this formula. These arc: (1) no beta-carotene; (2) lower amounts of zinc; and (3) no beta-carotene and lower amounts of zinc.¹ A supplement sold under the name TOZAL® Eye Health Formula includes lutein, zeaxanthin and omega-3 Fatty Acids (EPA & DHA), and antocyanosides. ¹. AGE-RELATED EYE DISEASE STUDY 2 PROTOCOL, version 2.1 Apr. 26, 2006

### SUMMARY

We have discovered a dietary supplement for eye health that suppresses not only oxidation, but also angiogenesis and inflammation. The supplement inhibits development and progression of a range of retinal diseases and degenerations, particularly AMD and diabetic eye disease. Supplements according to the invention can suppress oxidation, neo-vascularization and inflammation that promotes macular degeneration. They can also suppress the inflammation and neo-vascularization that promotes diabetic retinopathy and diabetic macular edema. Supplements according to the invention can also reverse some of the vision loss associated with these conditions.

The supplements are designed in unit dosages for periodic long-term administration, for example, once or twice daily for a period of at least one month and preferably six months or more. The supplements are designed to prevent, reduce, ameliorate, delay or reverse eye diseases, particularly age-related macular degeneration and diabetic eye diseases.

Stated most generally, one aspect of the invention features nutritional supplements comprising: an anti-oxidant component; an anti-inflammatory component; and an anti-angiogenic component.

Preferred anti-oxidants also include: vitamin E, including tocopherols and tocotrienols, epigallocatechin-3-gallate (EGCG), vitamin C, lutein and zeaxanthin.

Preferred anti-inflammatory agents include: green tea, catechins including epigallocatechin-3-gallate (EGCG) (also an antioxidant), d-alpha-tocopherol, and mixed tocopherols, and tocotrienols (also antioxidants), and omega-3 essential fatty acids, including EPA and DHA.

Preferred anti-angiogenic compounds include: polyphenols, including tocotrienol (also is an antioxidant); epigallocatechin-3-gallate (EGCG) (also an anti-oxidant and anti-inflammatory), resveratrol, daizein, genistein, flavnoids (such as quercetin, myrietin, fisetin, silibinin, kaempferol, 3-hydroxyflavone) and flavones (such as 3,4-dihydroxyflavone, flavone, flavopiridol, luteolin, genistein, apigenin and fisetin); isoflavones (such as daidzein, genistein and resveratrol); flavanones (such as eriodictyol and hesperetin), catechins such as catechin-3 gallate (CG), epicatechin-3 gallate (ECG), and, epigallocatechin (EGC) and epicatechin (EC) and omega-3 essential fatty acids.

In another aspect, the invention features either a green tea extract, a tocotrienol or (preferably) both, together with at least one omega-3 fatty acid and vitamin D. Preferably the nutritional supplement includes both green tea extract and tocotrienol. In this second aspect the supplement may include one or more of the above listed anti-inflammatory, anti-oxidant and anti-angiogenic compounds. Specifically preferred compounds for this aspect of the invention (in addition to tocotrienol and ,green tea extract) are lutein, zeaxanthin, vitamin C, vitamin D, and vitamin E (in the form of mixed tocopherols).

The details of one or more embodiments of the invention are set forth in the accompanying drawings and the description below. Other features, objects, and advantages of the invention will be apparent from the description and drawings, and from the claims.

### DETAILED DESCRIPTION

Without wishing to bind ourselves to a specific theory, we have considered the pathogenesis of macular degeneration, which involves three potentially harmful processes: inflammation, oxidation and neo-vascularization/angiogenesis (for the exudative form of macular degeneration and for proliferative diabetic retinopathy).

Preferred components of the invention are tocotrienols and green tea extract. The importance of these components for eye health are explained below. Preferably the composition includes lutein and zeaxanthin, which are naturally present in the macula. Also preferably, the composition includes omega-3 fatty acids, preferably long-chain omega 3 fatty acids, due to their anti-inflammatory and anti-angiogenic properties. Vitamin D is preferably included as an AMD preventative. Preferably a vitamin D serum level greater than 100 nmol/L, which typically requires a dosage of at least 4000 IU/day. Tocotrienols, a form of vitamin E with unsaturated hydrocarbon chains, are present as potent angiogenic inhibitors.

Below we discuss considerations in formulating the compositions generally, followed by a specific formulation that we presently prefer.

### Considerations Regarding Vitamin E

We have limited the amount of vitamin E, which, while useful in controlled doses (below 600 IU), may be harmful in very high doses.

“Vitamin E” refers to a group of lipid-soluble phenols, tocopherols and tocotrienols that possess an aromatic chromanol head and a 16-carbon tail. In contrast to tocopherols that contain a saturated side chain, tocotrienols contain an unsaturated side chain. Tocotrienols exist in four different forms or isomers, termed alpha-, beta-, gamma- and delta-tocotrienol, which contain different numbers of methyl groups on the chromanol ring. In homogeneous solution, the antioxidant potency of both tocopherol and tocotrienol is mainly proportional to the number of methyl groups on the aromoatic chromoal head, rather than the saturation of the 16-carbon tail. However in membranes the unsaturated tail of tocotrienols improves their mobility in the membrane bilayer, improving their ability to react with lipid radicals and recycling agents compared to tocopherols.

For this reason, tocotrienols are more powerful antioxidants than tocopherols. Levels of vitamin E in nutritional supplements are normally quantified in International Units (IU). However IUs refer only to levels of the d-alpha tocopherol vitamin E needed to prevent dietary deficiency of d-alpha tocopherol vitamin E, and does not reflect the biological potency and/or unique biological roles of beta-, gamma- and delta-tocopherols, or alpha-, beta-, gamma- and delta-tocotrienols.

Vitamin E is also anti-inflammatory. While d-alpha-tocopherol is anti-inflammatory, mixed tocopherols seems to have a more potent anti-inflammatory effect than d-alpha tocopherol alone (Reiter E, Jiang Q, Christen S Anti-inflammatory properties of alpha- and gamma-tocopherol. Mol Aspects Med (England), Oct-Dec 2007, 28(5-6) p 668-91). Tocotrienols also have an anti-inflammatory effect (Osiecki H, The role of chronic inflammation in cardiovascular disease and its regulation by nutrients. Ahern Med Rev (United States), Mar 2004, 9(1) p 32-53)

We combine 3 forms of vitamin E in this formulation: d-alpha-tocopherol; mixed tocopherols providing alpha-, beta- and gamma-tocopherol, and mixed tocotrienols. We claim between 50 and 200 IU of D-α-tocopherol, preferably about 153 IU of d-alpha-tocopherol and between 5 and 30 mg of mixed tocopherols, preferably about 20 IU of mixed tocopherols. The current formulation provides between about 25 to 60 mg of tocotrienols from palm oil, and preferably 30 mg of tocotrienols from palm oil, or between about 10 to 24 mg of tocotrienols, and preferably about 12.5 mg of tocotrienols from Tocomin SuproBio™ that provides for better absorption of tocotrienols, and therefore higher potency at a lower oral dose.

### Considerations Regarding Anti-Angiogenics

There are a number of anti-agronomic compounds that are present in foods. These include curcumin, epigallocatechin-3-gallate (EGCG) in green tea, n-acetylcysteine, 1-α-2,5-dihydroxyvitamin D3, conjugated linoleic acid, eicosapentaenoic acid, genistein, fisetin, quercetin, luteolin and resveratrol. All of these compounds can play a role in suppressing the neovasularization in age-related macular degeneration, diabetic retinopathy and diabetic macular edema.

Preferred methods for determining the anti angiogenic capacity of a substance include cell culture studies, and in vivo studies using two well-characterized angiogenic models—1) the mouse dorsal air sac (DAS) assay and 2) the chick embryo chorioallantoic membrane (CAM) assay. Tocotrienols are anti-angiogenic in these studies. They are also anti angiogenic in suppressing also fibroblast growth factor-induced proliferation, migration and tube formation in human umbilical vein endothelial cells (HUVEC), and γ-tocotrienol has the highest activity. γ-tocotrienol suppresses angiogenesis by suppressing the phosphorylation of phosophoinositide-dependent protein kinase and Akt (protein kinase B), a serine/threonine-specific protein kinase family, and by increasing the phosphorylation of apoptosis signal-regulating kinase and p38 in fibroblast growth factor-treated HUVEC). So the anti-angiogenic effects of γ-tocotrienol is due not only to suppression of growth factor signaling but also on the induction of apoptosis in endothelial cells (Nagagaw K, Shibata A, Yamashita S, Tsuzuki T, Kariya J, Oikawa S, Miyazawa T. “In vivo angiogenesis is suppressed by unsaturated vitamin E, tocotrienol”. J. Nutr. 137:1938-1943, 2007).

The current formulation provides between about 25 to 60 mg of tocotrienols from palm oil, and preferably 30 mg of tocotrienols from palm oil, or between about 10 to 24 mg of tocotrienols, and preferably about 12.5 mg of tocotrienols from Tocomin SuproBio™ that provides for better absorption of tocotrienols, and therefore higher potency at a lower oral dose.

Another aspect of anti-angiogenesis is control of endothelial cell proliferation. A mouse corena model for measuring that property is found in Brakenhielm, E., et al., “Suppression of angiogenesis, tumor growth and wound healing by resveratrol, a natural compound in red wine and grapes, FASEB J 15(2001) 1798-1800, particularly in FIG. 1. For example, it is desirable for the dietary supplement to exhibit the ability to reduce growth factor-induced endothelial cell proliferation (as measured in FIG. 1, e.g.) with IC₅₀ less than 2 μM.

### Green Tea

Epigallocatechin-3-gallate (EGCG), catechin-3 gallate (CG), epicatechin-3 gallate (ECG), and, epigallocatechin (EGC) and epicatechin (EC) are catechins found in green tea. EGCG is the predominant catechin in green tea. It has been shown to suppress endothelial cell growth in vitro in bovine endothelial cells stimulated by fibroblast growth factor-2 (FGF-2) and to block the formation of new blood vessels in the chick chorioallantoic membrane assay. Drinking green tea has been shown in mice to block corneal neovascularization stimulated by VEGF at serum concentrations in the range of 0.1 to 0.3 micromoles, levels that are achievable in humans by drinking two to three cups of green tea a day (Cao Y, Cao R. Angiogenesis inhibited by drinking tea. Nature 398, 381, 1999). Similarly, Jung and co-workers have shown that EGCG reduces microvessel density in a human colon cancer cell line by blocking the induction of VEGF. (Jung Y D, Kim M S, Shin B A et al. EGCG, a major component of green tea, inhibits tumour growth by inhibiting VEGF induction in human carcinoma cells. Br. J. Cancer 84, 844-850, 2001). It has also been shown, using the Matigel-induced tube formation assay, another widely used angiogenesis assay, that EGCG and CG almost completely abolished tube formation. The same group has shown that very low serum concentrations (0.01-1 microM) of EGCG, CG, and, to a lesser extent, ECG, inhibit VEGF by VEGF-dependent phosphorylation of VEGFR-2, the receptor most responsible for angiogenesis (Lamy S, Gingras D, Belivcau R. Green tea catechins inhibit vascular endothelial growth factor receptor phosphylation. Cancer Research 62, 381-385, 2002).

EGCG is a strong anti-oxidant, and has high affinity for the lipid layers of the cell membrane. EGCS is more effective than ascorbic acid (vitamin C) and tocopherol (vitamin E) in quenching reactive oxygen species (Zhao B L, Li X J, He R G, Cheng S J, Win W J. Savaging effect of extracts of green tea and natural antioxidants on active oxygen radicals. Cell Biophysics 14, 175-185, 1989). Green tea has been shown to significantly increase plasma total antioxidant status in humans (Serafina, M. Ghiselli A, Ferro-Luzzi A. In vivo antioxidant effect of green and black tea in man. Eur. J. Clin. Nutri. 50:28-32, 1996).

### Considerations Regarding Anti-Inflammatories; Green Tea as an Anti-Inflammatory

Inflammation causes changes to tissue architecture by alterating extracellular matrix via the activity of serine- and metallo-proteinases. Among the serine-proteinases, ECGC has been shown to inhibit leukocyte elastase that would otherwise disrupt the extracellular elastic network, (Sartor L, Pezzato E, Garbisa S. (-)Epigallocatechn-3-gallate inhibits leukocyte elastase: potential of the phyto-factor in hindering inflammation, emphysema and invasion. J Leukoc Bio 71:73-79, 2002 and also inhibits the metallo-proteases MMP-2 and MMP-9. (Garbisa S, Sartor L, Biggin S, Salvato B, Benelli R, Albini A. Tumor gelatinases and invasion inhibited by the green tea. Cancer 91:822-832, 2001).

Suganuma and co-workers have shown that ECGC and green tea block the gene expression of TNF-alpha and the cytokines IL-1 beta and IL-10 induced by TNF-alpha (Suganuma M, Sueoka E, Sueoka N, et al. Mechanisms of cancer prevention by tea polyphenols based on inhibition of TNF-alpha expression. Biofactors (Netherlands), 2000, 13(1-4) p 67-72).

The current formulation provides green tea extract providing between about 40 and about 350 mg of EGCG, or between about 55 to about 175 mg of EGCG, preferably containing about 120 mg of EGCG, or providing the components of the equivalent of between about ½ and about 6 cups of green tea, or between about 2 to about 3 cups of green tea, or preferably the equivalent of about 3 cups of green tea.

### Vitamin D

The formulation preferably includes between about 1000 IU to about 6000 IU of vitamin D, or between about 2000 IU to about 6000 IU of vitamin D, or between about 2000 and 5000 IU of vitamin D, or preferably about 4000 IU of vitamin D

### Omega Fatty Acids

The formulation includes between 450 to 650 mg of EPA, or between about 500 to about 600 of eicosapentaenoic acid (EPA) or preferably 567 mg of EPA. What is claimed in between about 200 mg to about 400 mg of docosahexaenoic acid (DHA) and preferably about 298 mg of DHA.

### Lutein and Zeaxanthin

Lutein preferably is present at 4 to 10 mg of Lutein, or 6 to 10 mg, or preferably about 8 mg of Lutein

Zeaxanthin preferably is present at about 1 to 3 mg of Zeaxanthin and preferably 2 mg.

### Vitamin C

Vitamin C preferably is present at between 250 and 750 mg of vitamin C, preferably 500 mg.

Preferably zinc is provided as zinc gluconate (because it is better absorbed than zinc oxide) at between about 3 and 15 mg of zinc gluconate, and preferably about 9 mg of zinc gluconate.

### Other Components

Copper gluconate is better absorbed when taken orally than copper oxide and preferably it is included in a ratio of zinc gluconate to copper gluconate of between about 35:1 to about 45:1, and preferably 36:1.

Preferably the composition has effectively no beta-carotene, which we believe is unnecessary because there is no beta-carotene in the retina and beta-carotene may be harmful.

We have included zinc due to its function in the immune system and in regulating inflammation, but again at controlled doses (below 16 mg).

### Specific Formulation

Without intending to limit the invention, we provide one particularly preferred embodiment, which includes two components, a tablet and two gel capsules. These components are packed together with instructions to take one tablet a day and 2 soft gels a day.

### Tablet

| Component | Amount | |
|---|---|---|
| Lutein (25%) | 8 | mg |
| Zeaxanthin (5%) | 2 | mg |
| Ascorbic Acid (97%) cellulose | 500 | mg |
| Zinc Gluconate (13%) | 9 | mg |
| Copper Gluconate (13%) | 0.25 | mg |
| EGCG in Green Tea Extract | 120 | mg |

| | |
|---|---|
| Inert ingredients² | — |

²Micro crystalline Celluose (Tabulose) 250 mg; Stearic Acid 35 mg; Croscamellose sodium (Solutab-Blanver, Accelerate-Stauber 8 mg; Silicon Dioxide (Flo-gard) 12 mg; Magnesium Stearate (vegetable grade) 12 mg.

Another component of this embodiment are two soft gel caps, together providing the following composition.

### Soft Gelcap

| Component | Amount | |
|---|---|---|
| Vitamin D3 | 4000 | IU |
| EPA (eicosapentaenoic acid) | 567 | mg |
| DHA (docosahexaenoic acid) | 298 | mg |
| Vitamin E; D-α-tocopherol | 153 | IU |
| CovioxT-70 ® mixed tocopherol | 20 | mg |
| concentrate | | |
| Tocomin ® Supra-Bio ™ (mixture of | 12 | mg |
| tocotrienols) | | |

| | |
|---|---|
| Inert ingredients³ | — |

³Microcrystalline cellulose, stearic acid, magnesium stearate, silicon dioxide, croscarmellose sodium, hydroxypropylmethyl cellulose, polydextrose, carboxymethyl cellulose, maltodextrin, dextrose monohydrate, talc FD&C Blue #2, titanium dioxide, mica medium chain triglycerides, lecithin.

A number of embodiments of the invention have been described. Nevertheless, it will be understood that various modifications may be made without departing from the spirit and scope of the invention. For example, all of the components can be combined in a single formulation, or the components may be separated into multiple formulations such as the gel cap and tablet described above. The components may be packaged together in various ways, including separate packages for daily dosage forms, or packages containing multiple dosages of each component separately packaged. The invention includes formulations for intraocular injection containing the components, specifically a) catechins such as catechin-3 gallate (CG), epicatechin-3 gallate (ECG), and, epigallocatechin (EGC) and epicatechin (EC), or b) tocotrienols or c) both catechins such as catechin-3 gallate (CG), epicatechin-3 gallate (ECG), and, epigallocatechin (EGC) and epicatechin (EC) and tocotrienol. Accordingly, other embodiments are within the scope of the following claims.

What is claimed is:

1. A nutritional supplement to aid eye health comprising, in combination,
an anti-oxidant component;
an anti-inflammatory component; and
an anti-angiogenic component.

2. A nutritional supplement to aid eye health comprising, in combination,
either green tea extract, a tocotrienol or both green tea extract and a tocotrienol; at least one omega 3 fatty acid; and
vitamin D.

3. The nutritional supplement of claim 2 comprising both green tea extract and a tocotrienol.

4. The nutritional supplement of claim 3 further comprising tocopherol.

5. The supplement of claim 1 in which the supplement comprises green tea.

6. The supplement of claim 1 in which the anti-oxidant component includes a tocotrienol, where having the following formula, 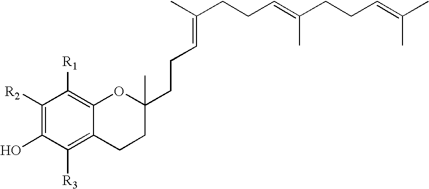
Where R1 R2 and R3 are defined below

| Type | R1 | R2 | R3 |
|---|---|---|---|
| α Tocotrienol | Me | Me | Me |
| β Tocotrienol | Me | H | Me |
| γ Tocotrienol | Me | Me | H |
| Δ Tocotrienol | Me | H | H |

7. The supplement of claim 1 or claim 2 in which the anti-angiogenic component includes at least one catechin.

8. The supplement of claim 7 in which the catechin is epigallocatechin gallate (EGCG), catechin-3 gallate (CG), epicatechin (EC), epicatechin gallate (ECG), epigallocatechin (EGC), or a combination of two or more of these compounds.

9. The composition of claim 1 or claim 2 in which the supplement inhibits endothelial cell proliferation with IC₅₀ less than 2 μM.

10. The composition of claim 2 further comprising vitamin C.

11. The composition of claim 2 further comprising lutein, zeaxanthin or both.

12. The composition of claim 1 in which the anti-oxidant component is selected from the group consisting of tocopherols and tocotrienols, epigallocatechin-3-gallate (EGCG), vitamin C, green tea extract, lutein and zeaxanthin.

13. The composition of claim 1 in which the anti-inflammatory component is selected from the group consisting of omega-3 essential fatty acids, including EPA and DHA, green tea, catechins including EGCG, d-alpha-tocopherol, mixed tocopherols, and tocotrienols.

14. The composition of claim 1 comprising
a) lutcin;
b) zeaxanthin;
c) at least one ω-3 fatty acid;
d) vitamin D; and
e) tocotrienol.

15. The composition of claim 2 or claim 14 comprising d-alpha-tocopherol in a dosage below 400 IU.

16. The composition of claim 2 or claim 14 further comprising zinc present in a dosage below 16 mg.

17. The composition of claim 2 or claim 14 wherein the ω-3 fatty acid is docosahexaenoic acid (DHA), eicosapentaenoic acid (EPA) or a combination of EPA and DHA.

18. The composition of claim 14 further comprising green tea.

19. The composition of claim 16 further comprising copper.

20. A composition comprising at least one soft gel cap and at least one tablet, said soft gel cap or soft gel caps comprising vitamin D and tocotrienol, and said tablet comprising green tea extract and lutcin, said soil gel cap or soft gel caps and said tablet being packaged together in a single package.

21. A method of preventing, reducing, delaying or reversing eye damage associated with age-related macular degeneration or diabetic eye disease, comprising administering to a patient in need thereof a composition in an amount effective to treat or reduce eye damage, said composition comprising an inflammatory agent, an anti-oxidant agent, and an anti-angiogenic agent.

22. A method of preventing, reducing, delaying or reversing eye damage associated with age-related macular degeneration or diabetic eye disease, comprising administering to a patient in need thereof a composition in an amount effective to treat or reduce eye damage, said composition comprising the components of the composition of claim 1 or claim 20.
